(19) United States
(12) Patent Application Publication
(10) Pub. No.: US 2013/0190742 A1
(43) Pub. Date: Jul. 25, 2013

(54) SYSTEM AND METHOD FOR MODIFICATION AND/OR SMOOTHING OF TISSUE WITH LASER ABLATION

(71) Applicants: Daniel Patrick Connors, Pleasant Valley, NY (US); Jerome Felsenstein, Ossining, NY (US); Robert E. Trzcinski, Rhinebeck, NY (US); James J. Wynne, Mt. Kisco, NY (US); Donna S. Zupanski-Nielsen, Yorktown Heights, NY (US)

(72) Inventors: Daniel Patrick Connors, Pleasant Valley, NY (US); Jerome Felsenstein, Ossining, NY (US); Robert E. Trzcinski, Rhinebeck, NY (US); James J. Wynne, Mt. Kisco, NY (US); Donna S. Zupanski-Nielsen, Yorktown Heights, NY (US)

(73) Assignee: INTERNATIONAL BUSINESS MACHINES CORPORATION, ARMONK, NY (US)

(21) Appl. No.: 13/640,588

(22) PCT Filed: Apr. 12, 2011
(86) PCT No.: PCT/US11/32061

§ 371 Date: Mar. 21, 2013

Related U.S. Application Data
(60) Provisional application No. 61/323,590, filed on Apr. 13, 2010.

(51) Int. Cl. A61B 18/20

(52) U.S. Cl. CPC A61B 18/20
USPC 606/17

(57) ABSTRACT

Disclosed is an improved system and method for efficiently removing tissue using laser ablation. A first laser emits a first laser beam with a variable first integrated fluence sufficient to ablate tissue. The first laser beam is movably positioned over one or more surfaces of the tissue and the first integrated fluence varies over different levels with position, so different thicknesses of tissue are ablated at different surface positions in order to modify the contour of the surface of the tissue. Modifications include tissue smoothing, removing, feathering, sharpening, and roughening. In one preferred embodiment the tissue is eschar that is removed, unveiling viable tissue. In alternate preferred embodiments, one or more additional lasers beams with different wavelengths, with integrated fluence sufficient to ablate tissue, are moved over the surface of the tissue until a second ablation reaches a second self-termination point, e.g., determined by affects of chemicals below the termination point that absorb the second laser beam without producing the temperature increase necessary for ablation to continue.

This application claims priority to provisionally filed patent application No. 61/323,590, filed on Apr. 13, 2010 and entitled “System and Method for Modification and/or Smoothing of Tissue with Laser Ablation.” This application is herein incorporated by reference in its entirety.

### FIELD OF THE INVENTION

This invention relates to the field of modifying the surface of human or animal tissue using laser ablation. More specifically, the invention relates to using laser ablation to modify, smooth, and/or remove necrotic tissue, like burn eschar, decubitus ulcers, and stasis ulcers, and/or remove foreign objects from tissue, in addition to eradicating infectious agents from the tissue being ablated.

### BACKGROUND OF THE INVENTION

Severe third degree burns produce burn eschar (char), which has to be debrided (removed) in order to uncover viable underlying tissue. This removal is required to facilitate healing and/or prepare the tissue to receive skin grafts. Typically severe burn eschar is currently removed using mechanical methods, e.g., removal by scalpel, abrasive methods, etc. These methods are slow, tedious, and traumatic, and often incompletely remove the eschar.

Similarly, decubitus, stasis, and neuropathic ulcers that form in skin wounds must be debrided to unveil viable underlying tissue, inorder to facilitate healing. Again, such necrotic tissue is currently removed using mechanical methods, with inevitable collateral damage to the underlying viable tissue. Further, such mechanical methods can make the tissue very susceptible to infection, inevitably causing excessive loss of viable underlying tissue, and most often resulting in excessive scarring.

Laser ablation of tissue is a process wherein the laser radiation is absorbed in a layer less than approximately 10 um at the surface of the tissue. The preferred wavelength for such strong absorption is in the ultraviolet region of the electromagnetic spectrum. This absorption converts the long chain protein molecules of the tissue into smaller, more volatile fragments, which are ejected from the surface, carrying away essentially all of the deposited laser energy in a plume composed of miniscule tissue fragments down to the molecular level, as well as water vapor and other gases. Such ablation constitutes tissue removal with minimal thermal damage to the underlying tissue, while destroying any remaining infectious agents, eliminating the sources of infection. These results are described in the publication Lane et al., “Ultraviolet-Laser Ablation of Skin,” Archives of Dermatology, Vol 121, pp. 609-617, May 1985. The invention of excimer laser surgery is described in U.S. Pat. No. 4,784,135, issued on Nov. 15, 1988. Both these references are herein incorporated by reference in their entirety.

### PROBLEMS WITH THE PRIOR ART

Mechanical tissue removal techniques are imprecise, traumatic, and create a pathway for infection of the wound area.

Current laser tissue removal systems cannot remove large thicknesses of tissue quickly without risking collateral damage to viable tissue around or under the removed tissue Infrared and visible lasers used to remove tissue create collateral damage (ablation or excessive heating) of viable tissue.

The prior art does not address removing burns and necrotic lesions that do not penetrate through the entire thickness of the skin.

In particular, carbon dioxide lasers emitting infrared radiation at 10.6 micrometers can ablate tissue, but such irradiation leaves a layer of collateral damage that is approximately 100 micrometers thick and will ultimately become necrotic if not removed.

Chemical agents used to debride tissue work slowly and inefficiently and can result in undesirable reactions in surrounding tissue.

### ASPECTS OF THE INVENTION

An aspect of this invention is an improved system and method for removing tissue using laser ablation.

An aspect of this invention is an improved system and method for removing tissue in a controlled and sterile way using laser ablation.

An aspect of this invention is an improved system and method for removing tissue using laser ablation with precise endpoint termination.

An aspect of this invention is an improved system and method for removing tissue using laser ablation with a precise, self terminated endpoint.

An aspect of this invention is an improved system and method for removing burn eschar using laser ablation with precise endpoint termination, so there is minimal or no collateral damage, particularly to viable tissue beneath the burn eschar.

An aspect of this invention is an improved system and method for smoothing tissue using laser ablation.

An aspect of this invention is an improved system and method for “feathering” tissue, i.e., changing the boundary between abnormal and normal tissue from sharp to gradual, using laser ablation.

An aspect of this invention is an improved system and method for roughening tissue, e.g., smooth scar tissue, using laser ablation.

An aspect of this invention is an improved system and method for removing tissue using laser ablation, where the removed tissue is of infectious origin, superficial cutaneous malignancies, devitalized or necrotic tissue (e.g., decubitus, stasis, and/or neuropathic ulcers), benign neoplasms, skin wrinkles, excessive scar tissue, and/or areas of skin discoloration.

An aspect of this invention is an improved system and method for treating psoriatic plaque, as well as vitiliginous patches.

An aspect of this invention is an improved system and method for removing material using laser ablation, where the removed material is an organic body imbedded in viable tissue.

### BRIEF DESCRIPTION OF THE FIGURES

FIG. 1 is a block diagram of one preferred system embodying the present invention.

FIG. 2 is a flow chart disclosing the steps performed by the present invention.

FIG. 3 is a flow chart of a laser ablation process.

### SUMMARY OF THE INVENTION

The present invention is a system and method of ablating undesired tissue by movably positioning a first laser beam over one or more surfaces of the tissue at one or more points of surface irradiation to remove the undesirable tissue. The undesirable tissue is in proximity to viable tissue. The first ablation stops after reaching a first termination point at which point a thin layer of undesirable tissue remains to insure that the viable tissue is not damaged by the first laser.

In one preferred embodiment, a second laser beam is moved over the undesired tissue after reaching a first termination point. The second laser beam ablates the thin layer of undesirable tissue until reaching a second termination point, where the unveiled tissue is viable tissue.

The invention is capable of modifying the surface of the tissue in various way including: removing, smoothing, roughening, sharpening, and feathering.

### DETAILED DESCRIPTION OF THE INVENTION

The present invention is an improved system and method for removing tissue using laser ablation. The system comprises a first laser beam source capable of emitting a first laser beam having a first wavelength within a first wavelength range, a variable first integrated fluence sufficient to ablate tissue, and a pulsed (not continuous) output. The system further comprises a first adjustor for adjusting the first integrated fluence. A first laser positioner manipulates the first laser beam to be movably positioned over one or more surfaces of the tissue at two or more points of surface irradiation, and the first integrated fluence at the points of surface irradiation vary as the first laser beam position changes, so that the first laser beam at a first integrated fluence level ablates one or more first layers of the tissue at one or more first points of surface irradiation and the first integrated fluence adjustor changes the first integrated fluence to one or more second integrated fluences that ablate one or more second layers of the tissue at one or more of the second points of surface irradiation in order to modify these contour of the surface of the tissue. In one preferred embodiment of the invention the tissue is burn eschar.

The term “integrated fluence” is defined as the total energy per unit area delivered to a point of surface irradiation. In the preferred embodiment, the laser output is pulsed, and the integrated fluence comprises the fluence/pulse (energy/unit area/pulse) multiplied by the pulse rate (Hz) multiplied by the dwell time (seconds) before the laser beam is moved to a new point of surface irradiation. The integrated fluence may be adjusted by varying any combination of the fluence/pulse, the pulse rate, and/or the dwell time.

In alternate preferred embodiments, the system further comprises one or more second laser beam sources capable of emitting second pulsed laser beams having second wavelengths within a second wavelength range and variable second integrated fluences appropriate for ablating tissue. After the first laser beam has reached a first termination point in ablating the tissue, the second laser beam is moved by a laser positioner (either the first laser positioner or another) to further ablate tissue until this second ablation reaches a second termination point. In another preferred embodiment, the second termination point is a self terminating point, where the self termination is determined by affects of chemicals below the termination point that absorb the second laser beam without producing the heat required for ablation to continue.

This invention discloses a system and method that uses laser ablation for rapid bulk removal of tissue with minimal or no collateral damage to surrounding or underlying viable tissue. Using this invention, tissue surfaces can be accurately and quickly modified in a sterile manner. Modifications include tissue removal, smoothing, sharpening, roughening, and feathering. In a preferred embodiment, the invention is used for rapid bulk removal and/or alteration of burn eschar or other necrotic tissue.

In alternative embodiments, this process is followed by laser irradiation at less then 200 nm, e.g., 193 nm radiation from an ArF excimer laser, for precise and self-terminating removal of thinner layers of the remaining tissue. By using this second laser for ablation, burn eschar or other necrotic tissue can essentially be totally removed, thereby unveiling undamaged and sterilized underlying viable tissue amenable to healing and, if medically appropriate, ready to accept skin grafts.

A multiple lasers embodiment can use two ablating lasers with different wavelengths in sequence. The first laser, for example with an wavelength of 308 nm, is used for accelerated debridement/ablation, followed by a second laser, e.g., with a wavelength of 193 nm, used for very precise and well controlled debridement/ablation of tissue (e.g., burn eschar, decubitus, stasis, and neuropathic ulcers, other lesions with necrotic areas), ablating ultrathin layers of material with each pulse.

Where relatively thick tissue is to be removed, the process of debridement starts with the longer wavelength laser, such as the 308 nm XeCl excimer laser, that considerably accelerates initial debridement/ablation. Use of the second shorter wavelength laser, such as the 193 nm ArF laser, is very desirable when debriding very close to the interface between burn eschar or other necrotic tissue and viable tissue, where the overall speed of debridement need not be so rapid.

The decision as to when to switch from the longer wavelength laser to the shorter wavelength (193 nm ArF excimer) laser will be determined by the termination point of the first laser ablation. This decision can be made by human judgment. However, in preferred embodiments, the first laser ablation termination point is determined automatically. A preferred way of determining the first laser ablation termination point is by measuring the optical signature (e.g., darkness, color/hue, surface roughness) of the thinned tissue (burn eschar.) For example, a CCD color camera will provide an image on a monitor and/or feed its signal into a device containing image processing software applications.

In the burn eschar example, when removing large areas of burn eschar from burn victims, it is important to minimize the time of the burn eschar removal procedure, because of the critical status of the burn victim. Therefore, a method is needed for automatically detecting when all of the eschar has been ablated from an area being irradiated by an ablating laser, whereupon the ablating laser beam(s) will be moved to irradiate an adjacent area of eschar.

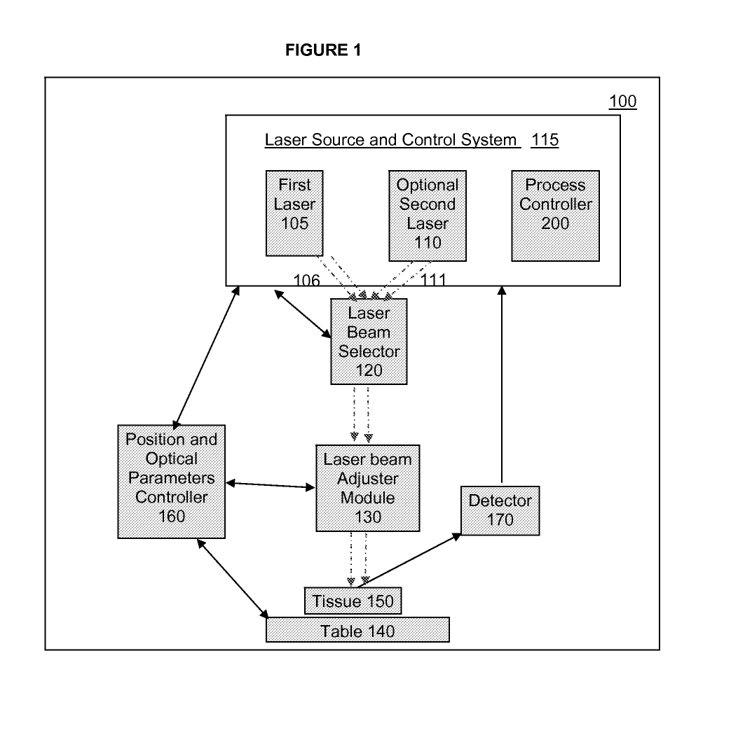

Now refer to FIG. 1, a block diagram of one embodiment of the present invention.

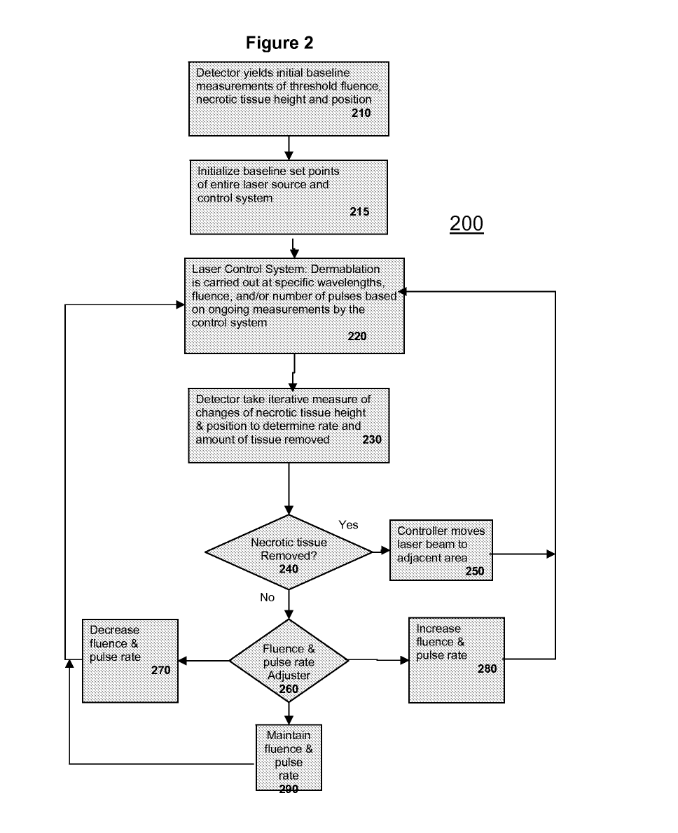

The system 100 comprises a first laser 105 emitting a laser beam 106 and, optionally, a second laser 110 emitting a laser beam 111. The integrated fluence of the laser beams are controlled by a laser source and control system 115. The laser system 115 also controls the positioning and optical parameters of the beams (106, 111) of each of the lasers, utilizing the process controller 200 (shown in one preferred embodiment in FIG. 2).

In some preferred embodiments, the system 100 has the ability to switch from the first laser 105 to the second laser 110 using a laser beam selector 120. The laser beam selector could be a set of mirrors. One of the mirrors could be a selecting mirror, which initially directs the output beam 106 of the first laser 105 into the laser beam adjuster module 130. When the selecting mirror changes position, the output beam 111 from the second laser 110 is directed to the laser beam adjuster module 130. Optical switch mechanisms like this are well known.

There are alternative ways to select the lasers 120. Both lasers could be directed through the laser beam adjuster module, but the laser reaching the laser output stage would be turned on while the other laser would be turned off. Alternatively, each of the laser beams (106, 111) could be controlled by a different manipulator in the laser beam adjuster module 130. In this embodiment, the laser beam selected by the laser switching module 120 would be directed to irradiate the surface of the tissue 150 by laser beam adjuster module 130.

In a preferred embodiment, the time to move or switch the laser beam irradiating the tissue is determined by an endpoint of ablation.

The position and optical parameters controller 160 controls the positioning of the beam output (106,111) of either of the lasers (105, 110) with respect to the portion of the surface of the tissue 150 being irradiated. The position and optical parameters controller 160 receives timing and positioning information from the laser source and control system 115, thereby controlling how long to irradiate an area (dwell time) at a given fluence per pulse and pulse rate and which area on the surface of the tissue to irradiate. The tissue can also be positioned with respect to the beam by moving a table 140 holding the tissue. The table position would also be controlled by the position and optical parameters controller 160 and can be used independently or in conjunction with the positioning of the beams of the lasers. The lasers (105, 110) and possibly the entire laser source and control system 115 can also be moved and positioned by mechanical apparatus like mechanical arms, navigators, and/or robots.

Position and optical parameters controllers 160 are well known. For example, in the medical field, mechanical arms, navigators, and surgical robots have been used for many years. See U.S. Pat. No. 5,086,401, Image-directed robotic system for precise robotic surgery including redundant consistency checking, to Glassman, et al.; U.S. Pat. No. 5,402,801, System and Method for Augmentation of Surgery to Taylor et al.; and U.S. Pat. No. 5,572,999, Robotic System for positioning a Surgical Instrument Relative to a Patient's Body to Funda, et al., which are in incorporated by reference in their entirety. In various embodiments, the surgical instruments in these references could either be the laser or the laser beam impinging on the surface of the tissue. Also see the product line of surgical robots sold and trademarked by Intuitive Surgical Corporation, e.g. the da Vinci Surgical System—http://www.intuitivesurgical.com/index.aspx.

The system 100 further comprises a detector 170, which measures one or more aspects of the ablation occurring at the surface of the tissue at a point of surface irradiation. This information is used by the laser source and control system 115 to: (i) change the fluence per pulse of the laser beam, (ii) change the pulse rate, (iii) change the pulse width, (iv) move the laser beam after a suitable dwell time to irradiate the tissue at a different location, and/or (v) switch to another laser beam. These changes and/or others affect the integrated fluence irradiating a given point on the tissue surface.

In a preferred embodiment, the first laser 105 has a wavelength (first wavelength) between 200 nanometers (nm) and 11 micrometers (mm) The preferred first wavelength is 308 nm. The working distance of the first laser 105, i.e., the distance between the output of the laser adjuster module 130 and surface of the tissue 150, is between 1 centimeter (cm) and 20 cm.

In a preferred embodiment, the integrated fluence of the first laser beam 106 is greater than 10 millijoules per centimeter squared. In a preferred embodiment, the fluence per pulse, pulse rate, dwell time, and/or pulse width of the laser beam are adjusted by the laser source and control system 115, using well known systems and methods.

The beam from the first laser can be continuous or pulsed. In a preferred embodiment, the beam is pulsed, and the duration of the pulse is in the range between 5 nanoseconds (ns) and 50 ns.

In a preferred embodiment, the first laser 105 beam 106 will ablate one or more layers of tissue at a given point of surface irradiation of the tissue 150, using a first level of integrated fluence until a first termination point is reached. In a preferred embodiment, the first termination point is determined by measuring an optical signature from the point of surface irradiation by the detector 170, in this case an optical detector 170. In general, the optical signature is light that is scattered or reflected from the tissue 150 during the ablation by the first laser 105 beam 106. Examples of optical signatures include: a degree of darkness, a change of color, a change in an amount of reflected light, a change in an amount of scattered light, and a change of surface roughness. One example of a detector 170 would be a camera system that has image processing functions to detect optical signatures. The detector would produce an output signal related to respective optical signature that would be provided to the laser source and control system 115.

In a preferred embodiment, the first termination point is the point at which the tissue 150 being ablated retains a thickness of necrotic tissue above viable tissue between 100 nm and 1 millimeter (mm) In a more preferred embodiment the first termination point is reached when the thickness of necrotic tissue 150 above viable tissue is 250 nm, and, more preferably, the necrotic tissue has a smooth surface. In certain preferred embodiments the desired termination point is achieved after most of the undesired tissue is removed but there is still enough undesired tissue remaining to protect the underlying (and/or adjacent) viable tissue so that there is little or no risk of causing damage to the viable tissue.

In certain preferred embodiments, the system and methods of the present invention are used to change the contours of the surface of tissue, e.g., by smoothing, roughening, or feathering. For example to produce cosmetic improvements to scar tissue, the surface contour of the ablated tissue is deliberately roughened. In other embodiments, the surface contour of the ablated tissue is “feathered,” where the tissue is thicker in one location and progressively gets thinner at locations progressively distant from the thickest location. In certain procedures in plastic and reconstructive surgery, feathering produces an improved appearance.

Variations in the thickness of the surface contour (smooth, rough, or feathered) after ablation are achieved by changing the integrated fluence of the first laser 105 beam 106 as the beam moves relative to the surface. To smooth the surface, the integrated fluence is higher when the surface of irradiation is elevated and the integrated fluence is lower when the surface of irradiation is relatively depressed.

The detector 170 determines which areas of the surfaces are relatively elevated or depressed.

In a preferred embodiment, the surface topography or roughness of the burn eschar or other tissue to be modified or removed (like necrotic tissue) is measured by illuminating the surface with an ultrashort light pulse (pulse width) from a pico- or femtosecond laser, the beam having a small spot size (i.e., small compared to the surface topographical features). As the beam is scanned over the surface of the tissue, the time it takes for the backscattered light to reach the detector, coupled with the known speed of light, will provide data that can be converted into a 3D map of the surface topography. This automated surface topographical technique may be overridden by the surgical practitioner, as needed. The topography of the surface at the point of irradiation provides feedback to the laser control system, which then adjusts the integrated fluence level of the first laser 105 beam 106, so that the elevated surfaces are ablated more than the depressed surfaces. In this way, the overall surface of the tissue is smoothed.

The laser beams can be scanned across the tissue surface in various ways. In one embodiment, when the surface is rough, the cross section of the first laser beam can be non-continuous, having two or more regions of higher integrated fluence separated by regions of lower integrated fluence, the fluence per pulse across the beam cross section being adjusted to map to the topography of the surface area being irradiated, so the higher integrated fluence irradiates more elevated areas of tissue and lower integrated fluence irradiates more depressed areas of tissue.

In another embodiment, the beam of the first laser has a continuous cross section with uniform fluence per pulse, and this beam is scanned continuously across the (necrotic) tissue, but with a variable scan rate (to change the integrated fluence) based on the necrotic tissue requirements, e.g. requirements for removal or other modification at the tissue location being scanned. In one preferred embodiment, this enables the integrated fluence irradiating a given area of tissue to be matched to the thickness of necrotic tissue to be removed.

In yet another embodiment, the beam of the first laser is scanned in a non-continuous manner, changing the integrated fluence over the scanned surface to avoid the irradiation of areas or regions of healthy viable tissue located adjacent to areas of (necrotic) tissue which is to be ablated.

In another embodiment, when the area of the (necrotic) tissue to be ablated is much larger than the laser beam cross section, the ablation mode may be “step and repeat”, i.e., an area of tissue that is the same size as the beam cross section is irradiated at a given integrated fluence; then the beam is moved to an adjacent area of tissue with minimal overlap, and the process is repeated with the appropriate integrated fluence; this process continues until all the particular tissue is ablated to a desired depth.

In a preferred embodiment, the tissue 150 being ablated is burn eschar. The invention provides a novel and efficient way of removing burn eschar, because initially a higher rate of ablation is performed which terminates upon reaching the first termination point. In this way most of the necrotic tissue is rapidly removed, but the viable tissue, e.g., below or adjacent to the removed tissue, is not damaged. Additionally, ablation by the first laser 105 can prepare the tissue 150 by reducing the thickness and/or modifying the surface contour of the tissue 150 so that a more precise removal can be performed by one or more second laser 110 beams 111. This embodiment provides an unparalleled capability to remove burn eschar, while automatically terminating the ablation when viable tissue is unveiled (in a preferred embodiment by the second laser 110), resulting in no collateral damage to the viable tissue, in addition to leaving it sterile and ready for further medical/surgical treatment, e.g., a skin graft.

In alternative embodiments, the first laser 105, followed by the second laser 110 (or even additional second lasers) as needed, can remove any of the following types of tissue 150:

Devitalized and necrotic tissue as in stasis ulcers, ecthymatous lesions, necrotizing fasciitis, neuropathic diabetic ulcers and decubiti;
ii) superficial cutaneous malignancies such as basal cell carcinoma, Bowens disease, lentigo maligna, squamous cell carcinoma;
iii) localized infectious lesions such as warts, molluscum contagiosum, superficial white and other types of onychomycosis, herpes simplex and zoster;

iv) benign lesions such as lentigenes, seborrheic keratoses, syringomata, xanthelasma, cutaneous polyps, actinic keratoses, hidrocystomata, sebaceous hyperplasia.

The tissue 150 may also be penetrated by organic objects (tissue), such as sea urchin spines, porcupine quills, splinters, insect parts, ticks, or tick parts, which may be removed by focusing the beam 106 from the first laser 105 to a small spot and scanning this beam around the periphery of the foreign object (tissue), ablating a minimum amount of tissue while freeing the object so that it may be easily extracted. If the foreign object (tissue) is penetrating dry tissue that contains a relatively low concentration of chloride ions, the second laser 110 beam 111 may be used to ablate such tissue.

In addition, other tissue 150 irregularities that can be modified by this preferred embodiment include superficial wrinkles, excessive scar tissue, and skin discoloration.

In alternative preferred embodiments, after a surface area of the tissue has been modified and the first termination point has been reached at all irradiation points on the surface for the first laser 105 beam 106, the second laser 110 beam 111 is selected by the laser selection/switching module 120.

In a preferred embodiment, the second laser 110 performs a finer ablation of tissue 150. For example, after the first laser 105 ablation leaves a smooth surface of burn eschar or other necrotic tissue approximately 250 nm thick, the second laser 110 can ablate this thinner layer of burn eschar or necrotic tissue with little variation in integrated fluence, removing much less tissue with each pulse than could be ablated by the first laser 105. In this manner, the removal of tissue close to viable tissue can be precisely controlled, and, even if a second termination point is missed, the damage to viable tissue, after the burn eschar has been completely removed at the tissue surface, would be minimal

In a preferred embodiment, the second laser 110 has a wavelength (first wavelength) between 180 nanometers (nm) and 10.6 micrometers (mm) In particular, the preferred second wavelength is 193 nm. The working distance of the second laser 110, i.e., the distance between the output of the laser 130 and surface of the tissue 150, is between 1 centimeter (cm) and 20 cm.

In a preferred embodiment, the integrated fluence of the second laser 110 beam 111 at the tissue 150 surface is greater than 10 millijoules per centimeter squared.

In a preferred embodiment, the integrated fluence of each laser is adjusted by the laser source and control system 115 using well known systems and methods.

The beam from the first laser can be continuous or pulsed. If the beam is pulsed, the duration of the pulse should preferably be in the range between 5 nanoseconds (ns) and 50 ns, and the fluence/pulse at the tissue 150 surface is greater than 10 millijoules per centimeter squared.

In a preferred embodiment, the second laser 110 beam 111 will ablate one or more layers of tissue from a given point of surface irradiation on the tissue 150 until a second termination point is reached.

In a preferred embodiment, the second termination point is automatically achieved due to the selection of the wavelength of the second laser 110 and the chemical characteristics of the viable tissue 150. In the preferred embodiments, the goal is to remove all of the necrotic tissue covering the viable tissue 150 and/or foreign material, while leaving the viable tissue unablated, and with no collateral damage. Because of the chemical and physical characteristics of viable tissue and the second wavelength of the second laser 110 being at or near 193 nm, the invention remarkably achieves this goal.

In a preferred embodiment, the second laser has a beam with a far ultraviolet (far-UV) 193 nm wavelength, being preferably an argon fluoride (ArF) pulsed excimer laser. Using this wavelength has the novel and unexpected result of being self terminating when viable tissue is exposed. Therefore, the second laser ablation termination point occurs automatically, without the need of a detection system. In this way, all burn eschar or other necrotic tissue can be removed with minimal or no collateral damage to the adjacent or underlying viable tissue, because once the second ablation laser contacts the viable tissue, the ablation process terminates.

Viable tissue differs from the burn eschar or other necrotic tissue in a very important way: the aqueous chloride ions in viable tissue are a strong absorber of ultraviolet radiation at wavelengths below 200 nm, with an absorption maximum at 190 nm. So the “salt water” that is a major component of viable tissue will “block” the incoming UV light and completely halt the ablation process.

The optical absorption spectrum of physiological saline solution shows extremely strong absorption at 193 nm. The mechanism of this absorption is the photodetachment of electrons from chloride ions, leaving chlorine atoms and solvated electrons dissolved in the aqueous medium. After each laser pulse, on a time scale that is very long compared to ablation and thermal diffusion times, the electrons will gradually encounter neutral chlorine atoms and recombine to form chloride ions, giving up the photodetachment energy to heat, but the heat will thermally diffuse into the surrounding tissue so that the temperature rise will be minimal and of no consequence to the viability or morphology of the underlying tissue. This phenomenon is described in detail in the publication Lane et al., “Ultraviolet-Laser Ablation of Skin, “Archives of Dermatology, Vol 121, pp. 609-617, May 1985.

In more detail, the 193 nm radiation generated by the ArF laser effectively ablates burn eschar or other necrotic tissue. (In a preferred embodiment, the thicker layers of burn eschar or other necrotic tissue are quickly removed by the first ablating laser.) When all of the eschar or other necrotic tissue in the field of the laser beam has been ablated, the exposed viable tissue, containing chloride ions dissolved in an aqueous environment (e.g., blood, blood plasma, lymph, moist viable tissue), will strongly absorb the 193 nm radiation without being ablated or thermally damaged. The absorption mechanism by chloride ions is a consequence of photodetachment of electrons from hydrated chloride ions. Basically, the energy from the 193 nm radiation strips electrons from the chloride ions, producing hydrated chlorine atoms and hydrated electrons. The 193 nm radiation is so depleted by this process that there is insufficient fluence irradiating the viable tissue to ablate or otherwise damage viable tissue.

In an alternative embodiment, an additional light source, having a wavelength specifically selected to detect chlorine atoms, can be introduced into the system. The specificity arises from the fact the chlorine atoms have well-defined electronic transitions that are only excited by specific wavelengths of light. Thus, the presence of chlorine atoms can be detected by observing the sudden increase of backscatter from this additional light source or by laser-induced fluorescence resulting from two-photon absorption of the additional light source. The detection of chlorine atoms provides a signal to terminate the irradiation of the area of tissue that is free of burn eschar or other necrotic tissue, which can be achieved by shuttering the ablating 193 nm laser beam or shifting it to a different location to irradiate an area of unablated burn eschar or other necrotic tissue that is to be removed by laser ablation. By using such an additional light source to detect chlorine atom concentration, ablating lasers with different wavelengths can be used and controlled by a system using the additional light source to provide the control signal, based on the detection of chlorine atom concentration. Well-defined one-photon electronic transitions in chlorine atoms are excited by light at the infrared wavelengths 838 nm and 859 nm. Suitable sources of light at these wavelengths are well known. In particular, the titanium-sapphire laser may be tuned to either of these two wavelengths, or a thermally tuned diode laser may be tuned to emit light at 838 nm or 859 nm, exactly resonant with these electronic transitions in chlorine atoms. A well-defined two-photon electronic transition in chlorine atoms is excited by light at the ultraviolet wavelength 233 nm. A suitable source of light at this wavelength is well known. In particular, frequency-quadrupled light (at 233 nm), derived from a titanium-sapphire laser or a thermally tuned diode laser emitting light at 932 nm, will excite two-photon laser-induced fluorescence at a wavelength that is characteristic of chlorine atoms. Such light can easily be detected by known means and spectrally separated from all other sources of light, permitting very sensitive detection of the initial appearance of chlorine atoms, an indicator that viable tissue has been unveiled.

There are other embodiments of the invention used to promote automatic self termination of the second laser ablation.

In one embodiment, aqueous solutions of sodium chloride (NaCl) or its derivatives, such as physiological saline solution, are infused with chloride ions (Cl⁻). Irrigation with such solutions beneath the burn eschar or other necrotic tissue will create an enhanced barrier, ensuring that the laser irradiation does not damage any underlying viable tissue. In a preferred embodiment, the saline solution is introduced subcutaneously, below the damaged tissue in the region between compromised and healthy tissue. The solution has a preferred concentration of approximately 1% NaCl, which is similar to the concentration of NaCl in blood.

Another alternative method to prevent shock in severely burned patients is to introduce hypertonic salt solution by rapid intravenous infusion within the first 24 hours. This procedure will hydrate the patient and increase the concentration of Cl⁻ in the healthy tissue under the burn.

A combination of such methods will yield better laser debridement of burn eschar or other necrotic tissue. This enhancement will aid in the removal of damaged tissue without cauterizing or otherwise damaging underlying viable tissue.

This technique is also self terminating when removing some foreign organic materials from viable tissue, because the ablation will terminate when the foreign matter is removed, exposing underlying viable tissue. This technique will work exceptionally well when irradiating with laser light at wavelengths below 200 nm, where the foreign material ablates at a high rate while viable tissue is protected by its perfusion of chloride ions.

There are other uses and applications of the invention other than burn eschar debridement. For example: Wounds, infected tissue, necrotic tissue, and foreign organic objects can be precisely removed using the 193 nm ArF excimer laser or longer wavelength lasers

Due to poor circulation from diseases such as diabetes and from excessive localized pressure (e.g., lying in bed for long periods of time without moving), people develop decubitus ulcers and superficial wounds. These ulcers and wounds are not easily healed by traditional medical methods, because it is very difficult to remove the necrotic tissue by conventional methods, e.g., “cold steel” debridement, without causing collateral damage which inhibits healing. Irradiation of such ulcers and wounds by 193 nm ArF excimer laser light will eradicate these lesions with great precision and no collateral damage, while sterilizing and not introducing foreign infectious microbes, with all of the previously described advantages.

The 193 nm ArF excimer laser will ablate tissue that has a viral infection, such as herpes simplex and zoster at the ocular surface, with great control, curing the patient of such infections with no collateral damage.

The longer wavelength lasers, in combination with the 193 nm ArF excimer laser, will remove sea urchin spines, porcupine spines, cactus spines, and splinters by focusing the laser beam down to a diameter comparable to or smaller than the diameter of the foreign object and scanning the focused spot of irradiation around the foreign object, minimizing the collateral damage to the adjacent tissue. Conventional mechanical methods require the removal of excessive adjacent tissue, because many of these foreign objects are structured with reverse barbs, which penetrate laterally into the adjacent tissue.

Certain aggressive organisms attack the skin and cause necrosis. The 193 nm ArF excimer can rapidly remove and destroy such organisms with great depth control, minimizing collateral damage.

It is a known fact that 308 nm light can be used to treat psoriatic plaque, as well as vitiliginous patches. The 308 nm XeCl laser is a preferred source of such light. By irradiating such psoriatic plaque with laser integrated fluence above the threshold for ablation, thickened psoriatic plaque can be removed, exposing underlying psoriatic erythematous patches, which can be treated by 308 nm light at subablative fluences.

Now refer to FIG. 2, a block diagram of one embodiment of the process controller 200 of the present invention. The process controller 200 comprises step 210, initial baseline measurements of laser ablation threshold fluence/pulse, e.g., determined by (necrotic) tissue height and position using the appropriate “detector” (e.g., photodetector, camera, sensor) and/or human judgment. These measurements 210 are used to carry out step 215, the initialization of the baseline set points of the entire system. Then, in step 220, ablation is performed by the laser system at specific wavelengths, fluence/pulse, dwell time, and/or number of pulses, based on ongoing measurements. As such, a desired integrated fluence can be determined for each type of tissue for a particular ablation rate for the respective tissue. In step 230, iterative measurements are taken of the changes in the (necrotic) tissue height and position, using the “detector 170” and/or human judgment to determine the rate and amount of tissue removed. In step 240, an assessment is made by the process controller 200 and/or human judgment as to whether the (necrotic) tissue is sufficiently ablated. If the assessment is positive, i.e., Yes, the position and optical parameters controller 160 will move the laser beam to an adjacent untreated area of tissue, initiating ablation as in step 220. If the assessment is negative, i.e., No, the position and optical parameters controller 160 will adjust 260 the integrated fluence after the determination is made whether to go to step 270, 280, or 290. In step 270, the integrated fluence is decreased and ablation continues as in step 220, but at a reduced rate. Step 270 may also determine if the laser ablation should be terminated. In step 280, the integrated fluence is increased and ablation continues as in step 220, but at an increased rate. In step 290, the integrated fluence parameters are not changed and ablation continues as in step 220 at the same rate. This process flow continues until the desired amount of (necrotic) tissue is ablated, either automatically under the control of the process controller 200 or with the discretion of human judgment.

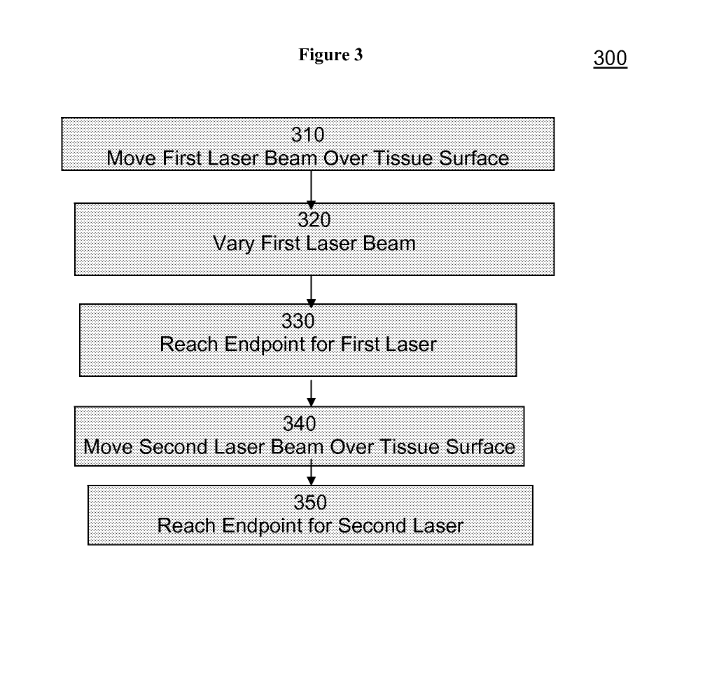

FIG. 3 is a flow chart of a laser ablation process 300.

In step 310, a first laser beam 106 is movably positioned by system 100 to irradiate one or more areas of tissue surface. The first laser beam ablates undesired tissue that is in proximity to viable tissue until reaching a first termination point. As described above, in a preferred embodiment the first termination point is the point at which a thin layer of undesirable tissue remains, to insure that the viable tissue is not damaged by the first laser beam 106. In other preferred embodiments, e.g. where non-necrotic tissue is being removed, the first termination point might be determined by a depth of tissue removal.

As described above, in many preferred embodiments, the first laser beam is adjusted as the beam is moved. In step 320, the first laser beam integrated fluence is varied as the beam moves, in order to change the effect of the first laser on one or more of the points of surface irradiation. Any other variation that can be used to affect the rate at which the first laser beam 106 ablates tissue can also be changed.

In some preferred embodiments of step 320, the first laser beam varies at the points of surface irradiation as the first laser beam moves, wherein the first laser beam first ablates one or more layers of the tissue at one or more of the points of surface irradiation that have a higher elevation with a higher ablation rate and the first laser beam varies to ablate one or more of the points of surface irradiation that have a depressed elevation with a lower ablation rate, so that the surface of the tissue becomes smoother.

In step 330, when the termination point is reached for the first laser beam 106, the application of the first laser beam is terminated as described above.

In some preferred embodiments, the optional step 340 is practiced. In step 340, a second laser is applied by system 100, after the first termination point is reached and the undesirable tissue is reduced to a thin layer. The system 100 moves the second laser beam 111 so that the second laser beam ablates the remaining thin layer of undesirable tissue. This second ablation of undesirable tissue continues until reaching a second termination point, where the unveiled tissue is viable tissue. At this point (step 350) the second ablation terminates. In some embodiments, as described above, the termination is automatic. At the second termination point, viable tissue is unveiled with minimum or no damage caused by either the first or second laser beams (106, 111.)

Given this disclosure, one skilled in the art could envision different and alternative embodiments of this invention which would be considered within the scope of the invention. As a non-limiting example, the invention could use more than one second laser at different locations on the surface of the tissues or could use more than one second laser to change ablation rates and/or end point determination.

1-90. (canceled)

91. A system for ablating undesirable tissue with a non uniform surface comprising:
a first laser beam source capable of emitting a first laser beam having a first wavelength within a first wavelength range and a variable first integrated fluence sufficient to ablate undesirable tissue;
a first integrated fluence adjustor for adjusting the first integrated fluence;
a first laser positioner that manipulates the first laser beam so that the first laser beam is movably positioned over one or more surfaces of the undesirable tissue at one or more points on the surface; and
wherein the first fluence adjustor is capbable of adjusting the first integrated fluence at two or more of the points as the first laser beam position changes and the first laser beam is capable of ablating one or more first layers of the undesirable tissue with a greater thickness at one or more of the first points at a first fluence level and the first fluence adjustor is capable of changing the first integrated fluence to one or more second fluence levels to ablate one or more second layers of the undesirable tissue with a lesser thickness at one or more second points in order to modify the contour of the surface of the undesirable tissue where the first fluence level has a higher aggregated fluence than the second fluence level so that the greater thicknesses of the undesirable tissue are ablated more than the lesser thicknesses.

92. A system, as in claim 91, where the undesirable tissue at the first points and second points is ablated so that the first points and second points become more co-planar.

93. A system, as in claim 91, where the thickness of the undesirable tissue to be ablated is between 100 nm and 1 centimeter of thickness.

94. A system, as in claim 91, where the undesirable tissue is any one of the following classes:
i) devitalized and necrotic tissue as in stasis ulcers, ecthymatous lesions, necrotizing fasciitis, neuropathic diabetic ulcers and decubiti;
ii) superficial cutaneous malignancies such as basal cell carcinoma, Bowens disease, lentigo maligna, squamous cell carcinoma;
iii) infectious lesions such as warts, molluscum contagiosum, superficial white and other types of onychomycosis, herpes simplex and zoster;
iv) benign lesions such as lentigenes, seborrheic keratoses, syringomata, xanthelasma, cutaneous polyps, actinic keratoses, hidrocystomata, sebaceous hyperplasia;
v) eschar tissue and burn eschar tissue;
vi) an embedded organic foreign object; and
vii) superficial wrinkling, excess scar tissue, psoriatic plaque, and skin discoloration.

95. A system, as in claim 91, where the undesirable tissue is scar tissue and the modification to the contour is one or more of the following: thinning, smoothing, roughening, sharpening, and feathering.

96. A system, as in claim 91, where the first wavelength range is between 200 nanometers and 11 micrometers.

97. A system, as in claim 91, where the first ablation removes necrotic tissue and terminates at a protective endpoint that leaves a protective thickness of tissue of at least 250 nm of thickness below which there is viable tissue.

98. A system, as in claim 97, where the protective thickness is between 250 nm and 1 mm.

99. A system for ablating undesirable tissue with a non uniform surface comprising:
a first laser beam source capable of emitting a first laser beam having a first wavelength within a first wavelength range and a variable first integrated fluence sufficient to ablate tissue;
a second laser beam source capable of emitting a second laser beam having a second wavelength within a second wavelength range, and a second integrated fluence sufficient to ablate undesirable tissue;
one or more adjustors for adjusting one or more of the first integrated fluence and the second integrated fluence; and
one or more laser positioners capbable of manipulating the first laser beam so that the first laser beam is movably positioned over one or more surfaces of the undesirable tissue at one or more location points and the adjustor capable of adjusting the first integrated fluence of the first laser beam so that the first integrated fluence is greater at the location points where the undesirable tissue is thicker and the first integrated fluence is less at location points where the undesirable tissue is thinner, the first adjustor capable of turning off the first laser beam when a first termination point is reached, the laser positioner further capable of positioning the second laser beam after reaching the first termination point, so that the second laser beam can ablate a protective thickness of the undesirable tissue until reaching a second termination point where the unveiled tissue is viable tissue.

100. A system, as in claim 99, where one or more of the adjustors is capable of adjusting adjusting the one or more of the first integrated fluence and the second integrated fluence by varying one or more of the following: number of pulses, pulse width, fluence, pulse rate, and dwell time.

101. A system, as in claim 99, where a working distance between one of the first and second lasers and the surface of the undesirable tissue is between 1 cm and 20 cm.

102. A system, as in claim 101, where the second termination point is automatically determined by an absorption agent that absorbs sufficient energy of the second laser beam to prevent damage to the viable tissue.

103. A system, as in claim 102, where the absorption agent is one or more of the following: a dissolved salt, a dissolved ion, photo-detachment of the electron of the dissolved ion, a dissolved halide salt, a substance in suspension, a chloride ion, one or more ions naturally occurring in the viable tissue layer, a saline solution infused beneath the undesirable tissue layer, a saline solution administered subcutaneously, a physiologic saline solution, an aqueous NaCl solution in a concentration range between 3-25 grams of NaCl per liter of water, a saline solution introduced by intravenous infusion.

104. A system, as in claim 99, where a cross section of the second laser beam is non continuous, having two or more regions of higher integrated fluence separated by regions of lower integrated fluence.

105. A system, as in claim 99, further comprising an optical signature detector capable of terminating one or more of the first and second ablation upon detecting an optical signature.

106. A system, as in claim 105, where the optical signature is one or more of the following: a change in an amount of scattered light, a change in the amount of reflected light, a change in the spectral distribution of the scattered/reflected light from the incident light illuminating the area being ablated, a scattered light that increases because of the appearance of a new atomic species produced by the second wavelength irradiating the unveiled viable tissue, and an excitation of a chlorine atom being radiated by the second wavelength of one of 193 nm and approximately 233 nm.

107. A method of ablating undesirable tissue comprising the steps of:
movably positioning a first laser beam over one or more surfaces of the undesirable tissue at one or more points on the surface to remove the undesirable tissue that is in proximity to viable tissue until reaching a first termination point where a thin layer of undesirable tissue remains to insure that the viable tissue is not damaged by the first laser and further where more undesirable tissue is removed at the points where the undesirable tissue is thicker and less undesirable tissue is removed at points where the undesirable tissue is thinner.

108. A method, as in claim 107, where the undesirable tissue is thinner and points on the thinner, undesirable tissue are more co-planar.

109. A method, as in claim 107, further comprising the step of:
moving a second laser beam over the surface after reaching a first termination point so that the second laser beam ablates the remaining layer of undesirable tissue until reaching a second termination point where the unveiled tissue is viable tissue.
